(19) United States
(12) Patent Application Publication
(10) Pub. No.: US 2005/0153280 A1
(43) Pub. Date: Jul. 14, 2005

(54) Viral propagation system and uses thereof

(76) Inventor: Chiaho Shih, Houston, TX (US)

Correspondence Address: Benjamin Aaron Adler;ADLER & ASSOCIATES, 8011 Candle Lane, Houston, TX, 77071, US

(21) Appl. No.: 10/988,348

(22) Filed: Nov. 12, 2004

Related U.S. Application Data
(60) Provisional application No. 60/519,382, filed on Nov. 12, 2003.

(51) Int. Cl. C12Q 1/70; C12N 15/85; C12N 15/86

(52) U.S. Cl. 435/5; 435/455; 435/456

(57) ABSTRACT

Trans-differentiation induced by dexamethasone with or without oncostatin M results in cells that are capable of propagating and replicating hepatitis virus. Such trans-differentiated cells are useful for screening drugs that may affect the propagation and replication of hepatitis virus such as hepatitis B virus or hepatitis C virus.

### CROSS-REFERENCE TO RELATED APPLICATION

This non-provisional patent application claims benefit of provisional patent application U.S. Ser. No. 60/519,382, filed Nov. 12, 2003, now abandoned.

### FEDERAL FUNDING LEGEND

This invention was produced in part using funds obtained through National Institutes of Health grants RO1 CA70336 and CA84217. Consequently, the federal government has certain rights in this invention.

### BACKGROUND OF THE INVENTION

1. Field of the Invention

The present invention relates generally to the fields of virology and cell biology. More specifically, the present invention relates to a novel system for viral propagation.

2. Description of the Related Art

Among the many hepatoma cell lines, Huh7 and HepG2 are the most commonly used for the study of hepatitis B virus. This is because many liver cell lines do not support hepatitis B virus replication, despite the fact that they could exhibit several liver specific markers. In general, it is believed that well-differentiated hepatocytes are more likely to support hepatitis B virus replication, while dedifferentiated hepatocytes are less likely to support HBV replication. Transcription factors and chaperons are supposed to be important host factors for hepatitis B virus replication.

It was demonstrated more than two decades ago that a single dose of pancreatic carcinogen resulted in liver cell-like foci in the pancreas of hamsters. Long term treatment with ciprofibrate in rats can also induce trans-differentiation from pancreas to liver. Interestingly, when rats were fed a copper-depletion diet, followed by repletion with normal diet, it resulted in oval-like cells in pancreas. These pancreatic hepatocytes contained several common markers for hepatocytes. When these pancreatic hepatocyte-like cells were transplanted into a recipient mouse, they developed into foci of mature hepatocytes. Islet hepatocytes have also been observed in transgenic mice expressing keratinocyte growth factor in beta-cells. Indeed, pancreas and liver are closely related to each other embryologically. Taken together, these reports strongly suggest that pancreatic cells have the potential to convert into hepatocyte-like cells during chronic injury and regeneration.

AR42J cell line is from a rat pancreatic tumor induced in vivo by azaserine. It has been shown recently that a subclone of AR42J, AR42JB13, can trans-differentiate into hepatocyte-like cells when treated with dexamethasone in tissue culture. Oncostatin M, in the presence of dexamethasone, can facilitate this process. The trans-differentiated hepatocytes exhibited liver markers by immunofluorescence staining, including glutamine synthetase, α-1-antitrypsin, transferrin, and transthyretin. Furthermore, these cells can synthesize acute phase proteins and display detoxification activity.

The prior art is deficient in cells that will propagate and replicate viruses such as hepatitis viruses, including but not limited to hepatitis B virus and hepatitis C virus. The present invention fulfills this long-standing need and desire in the art by demonstrating that the trans-differentiated pancreatic hepatocytes can function like bona fide liver cells and support hepatitis B virus replication.

### SUMMARY OF THE INVENTION

The present invention provides a method of using trans-differenatiated cells to screen for drugs that affect propagation and replication of hepatitis virus such as hepatitis B or C virus. The trans-differentiation is induced by a glucocorticoid steroid or a glucocorticoid steroid plus an IL-6 homologue. A representative glucocorticoid steroid is dexamethasome, whereas a representative IL-6 homologue is oncostatin M. In one embodiment, the trans-differentiated cells are capable of propagating and replicating hepatitis B virus, as well as secreting hepatitis B surface antigens and hepatitis B e/core antigens. The screening method involves examining viral replication in the trans-differentiated cells in the presence or absence of a test compound, wherein inhibition of DNA replication or RNA transcription of hepatitis virus would indicate such test compound has anti-hepatitis virus activity.

In another embodiment, there is provided a transdifferentiated cell capable of propagating and replicating hepatitis B or hepatitis C virus.

In yet another embodiment, there is provided a method of transdifferentiating a first cell into a second cell capable of propagating a virus by contacting the cell with a glucocorticoid steroid with or without an IL-6 homologue.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIGS. 1A-B show stable hepatitis B virus-transfected clones B13-1 and B13-28 can be induced to secrete HBeAg (FIG. 1A) and HBsAg (FIG. 1B) by dexamethasone (Dex) and oncostatin M (OSM). The ELISA assay for HBsAg and HBeAg was performed as described elsewhere (Tai et al., 2002). The values and standard deviations are from a total of 4 independent induction experiments.

FIGS. 2A-B show immunofluorescence staining of HBV core antigen and liver-specific glutamine synthetase in trans-differentiated B13-1 (FIG. 2A) and B13-28 cells (FIG. 2B). Cells were treated with dexamethasone and oncostatin M for 7 days, followed by dual immunostaining with anti-HBc (green) and anti-glutamine synthetase (red).

Panel a, anti-HBc; b, anti-glutamine synthetase; c, overlaid image of a and b; d, differential interference contrast (DIC); e, uninduced control stained with anti-HBc; f, DAPI staining for nuclei of the same field as e. Panel g, anti-HBc; h, anti-glutamine synthetase; i, overlaid image of g and h; j, DIC; k, uninduced control, anti-glutamine synthetase; 1, DAPI staining for nuclei of the same field as k.

FIGS. 3A-B show immunofluorescence staining of HBV surface antigen and liver-specific transferrin in trans-differentiated B13-1 (FIG. 3A) and B13-28 cells (FIG. 3B). B13-1 cells were treated with dexamethasone and oncostatin M for 5 (panels a-d) or 7 (panels e-h) days, then immunostained with anti-HBs (green) and anti-transferrin (red). B13-28 cells were treated with dexamethasone and oncostatin M for 5 (panels i-1) or 7 (panels m-p) days, then immunostained with anti-HBs (green) and anti-transferrin (red).

FIG. 4 shows co-localization patterns of liver enriched transcription factors with HBsAg in the trans-differentiated B13-28 cells. Cells were treated with dexamethasone and oncostatin M for 7 days, followed by dual immunostaining. Upper panel: a, anti-C/EBP α(green); b, anti-HBs (red); c, overlaid image of a and b. Middle panel: d, anti-C/EBP β(green); e, anti-HBs (red); f, overlaid image of d and e. Lower panel: g, anti-HNF 4a (green); h, anit-HBs (red); i, overlaid image of g and h.

FIG. 5 shows Western blot analysis of liver, pancreas and HBV core protein expression in uninduced and induced B13-1 and B13-28 cells.

FIG. 6AB shows production of HBV small envelope-specific mRNA (preS2/S) can be significantly stimulated by dexamethasone and oncostatin M. FIG. 6A shows that twenty-five micrograms of total RNA from each sample were analyzed by Northern blot using a vector-free 3.1 kb hepatitis B virus DNA probe. Hepatitis B virus RNA from Qs21 was included as a positive control. Major hepatitis B virus-specific transcripts are indicated by arrows. 18S and 28S rRNA are shown below as an internal control.

FIG. 6B shows primer extension analysis revealed the preferential induction of pre-core specific RNA by dexamethasone and oncostatin M. Thirty micrograms of total RNAs, which were isolated from the B13-28 cells with or without dexamethasone and oncostatin M for 7 days, were used as the template for primer extension analysis. The extended products corresponding to pre-core and core specific RNAs are indicated by arrows.

FIGS. 7A7C shows intracellular hepatitis B virus DNA replication in B13-1 and B13-28 cells was significantly increased upon treatment with dexamethasone and oncostatin M. The cells from each 10-cm dish were harvested at different time points after treatment with dexamethasone and oncostatin M for 3, 5, and 7 days. Each lane was loaded with hepatitis B virus DNA extracted from each dish. Qs 21 is an hepatitis B virus-producing cell line and was included here as a positive control; 0 day, cells were cultured without dexamethasone and oncostatin M for a few days; RC, relaxed circle; SS, single-strand hepatitis B virus DNA replicative intermediates. A 3.1-kb hepatitis B virus DNA was used as a probe.

FIG. 7B shows Southern blot analysis of covalently closed circular (ccc) HBV DNA in the trans-differentiated B13-1 and B13-28 cells. The ccc DNA was extracted from cells grown with dexamethasone and oncostatin M for 7 days.

FIG. 7C shows Southern blot analysis of extracellular HBV DNA in the media of trans-differentiated B13-28 cells. Conditioned media were collected on days 5 and 7 post-induction. After centrifugation through a 20% sucrose cushion, resuspended pellets of hepatitis B virus particles were separated by isopycnic centrifugation through a cesium chloride gradient (20 to 50%). Fractions corresponding to the enveloped Dane particles (fractions 10 to 16, density=1.24 g/cm³) were pooled and extracelluar hepatitis B virus DNA was extracted and subjected to Southern blot analysis.

FIG. 8 shows electron microscopic examination of secreted HBV viral and subviral particles in the medium of B13-28 cells induced with dexamethasone and oncostatin M for 7 and 9 days. A, Spherical subviral particles; B, filamentous subviral particles; C, 42-nm Dane-like particles.

FIGS. 9A-D shows continuous presence of dexamethasone and oncostatin M is required for hepatitis B virus replication and gene expression in B13-1 and B13-28 cells. Both B13-1 and B13-28 cells were treated with dexamethasone and oncostatin M for 7 days. They were then cultured in medium without dexamethasone and oncostatin M for 7 days. Conditioned media were collected for the ELISA assays of HBsAg and HBcAg (FIG. 9A) and viral DNAs were harvested and subjected to Southern blot analyses (FIGS. 9B and C). FIG. 9D shows Western blot analysis for the proteins of HBcAg, α1-anti-trypsin, α-amylase, HBsAg, and α-tubulin.

### DETAILED DESCRIPTION OF THE INVENTION

Despite the existence of a large number of hepatoma cell lines, only a very limited number (HepG2, Huh7 and rat hepatoma 7777) support efficient replication of human hepatitis B virus (HBV). Recently, a rat pancreatic cell line (AR42J-B13) was shown to trans-differentiate to liver-like cells upon induction with dexamethasone (Dex). To determine if these hepatocytes can indeed function like bona fide liver cells and support replication of hepatotropic HBV, AR42J-B13 cells were stably transfected with hepatitis B virus DNA. Viral activities as well as host liver cell markers were examined with or without induction. A full spectrum of hepatitis B virus replicative intermediates, including covalently closed circular (ccc) DNA, can be detected in this system only after induction. Strikingly, the small envelope protein and RNA of hepatitis B virus were increased by 40- to 100-fold upon induction. In contrast, the level of HBV core antigen (HBcAg) specific RNA was not affected by induction, despite the fact that the protein level of HBcAg was dramatically increased as detected by Western blot and immunofluorescence microscopy. These results suggest a novel translational or post-translational control of HBcAg in this trans-differentiation system. Characteristic Dane particles and subviral particles were identified by electron microscopy. Continuous presence of dexamethasone is required for the maintenance of hepatitis B virus replication and gene expression.

In summary, HBV replication can be induced synchronously and maintained by physiological inducers in a tissue culture model of trans-differentiation. This novel system offers an opportunity for drug screening and molecular dissection of virus-host interaction at transcriptional and post-transcriptional levels. Moreover, this system may be useful for viral replication, morphogenesis, and virion release of other kinds of hepatitis viruses.

The present invention provides a method of using trans-differenatiated cells to screen for drugs that affect propagation and replication of hepatitis virus. The screening method involves examining viral replication in the trans-differentiated cells, wherein inhibition of DNA replication or RNA transcription of hepatitis virus in the presence of a test compound would indicate such test compound has anti-hepatitis virus activity. In general, the screening is applicable to hepatitis viruses including but not limited to hepatitis B virus or hepatitis C virus.

Preferably, the trans-differentiation is induced by a glucocorticoid steroid or a glucocorticoid steroid plus an IL-6 homologue. A representative glucocorticoid steroid is dexamethasome, whereas a representative IL-6 homologue is oncostatin M. In one embodiment, the trans-differentiated cells are capable of propagating and replicating hepatitis B virus, as well as secreting hepatitis B surface antigens and hepatitis B core antigens. Representative trans-differentiated cells include rat AR42J-B13-1 cells, AR42J-B13-10 cells, AR42J-B13-18 cells, and AR42J-B13-28 cells.

In another embodiment, there is provided a transdifferentiated cell capable of propagating and replicating hepatitis B or hepatitis C virus. Such transdifferentiated cell is also capable of secreting hepatitis B surface antigens and hepatitis B core antigens. The transdifferentiation is induced by a glucocorticoid steroid with or without an IL-6 homologue. Preferably, a representative glucocorticoid steroid is dexamethasome, and a representative IL-6 homologue is oncostatin M.

In yet another embodiment, there is provided a method of transdifferentiating a first cell into a second cell capable of propagating a virus. The cell is transdifferentiated by a glucocorticoid steroid with or without an IL-6 homologue. In one embodiment, the first cell is a pancreatic cell, and the second cell is a hepatic cell. Representative viruses include hepatitis B virus and hepatitis C virus. A representative glucocorticoid steroid is dexamethasome and a representative IL-6 homologue is oncostatin M.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the cells and methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

### EXAMPLE 1

Stable Transfection of AR42JB13 Cell Lines With Human Hepatitis B Virus DNA

AR42J-B13 cells (Shen et al., 2000; Mashima et al., 1996) were maintained in Dulbecco's modified Eagle's medium (low glucose 1 g/L) (GIBCO) containing penicillin, streptomycin and 10% fetal bovine serum) at 37° C. in an atmosphere of 5% CO₂. Dexamethasone (Dex) and oncostatin M (OSM) were prepared as described previously (Shen et al., 2000). Stable transfectants of AR42J-B13 cells were generated using a tandem dimer of HBV DNA (ayw subtype) in a pSV2Neo vector (Shih et al., 1989). Before transfection, AR42J-B13 cells were either treated or not treated with Dex+OSM for 7 days. Two micrograms of plasmid DNA were transfected using the FuGENE 6 transfection protocol (Roche). Stable transfectants were selected in medium containing 1 mg/ml G418 (Life Technologies). After 4-7 weeks, clones were picked, expanded and maintained initially in medium containing G418. Subsequently, clones B13-1 and B13-28 have been passaged in the absence of G418 for 18 months.

### EXAMPLE 2

Detection of HBV Surface Antigen (HBsAg) and e Antigen (HBeAg) In The Medium of B13-1 and B13-28 Cells By ELISA Assay

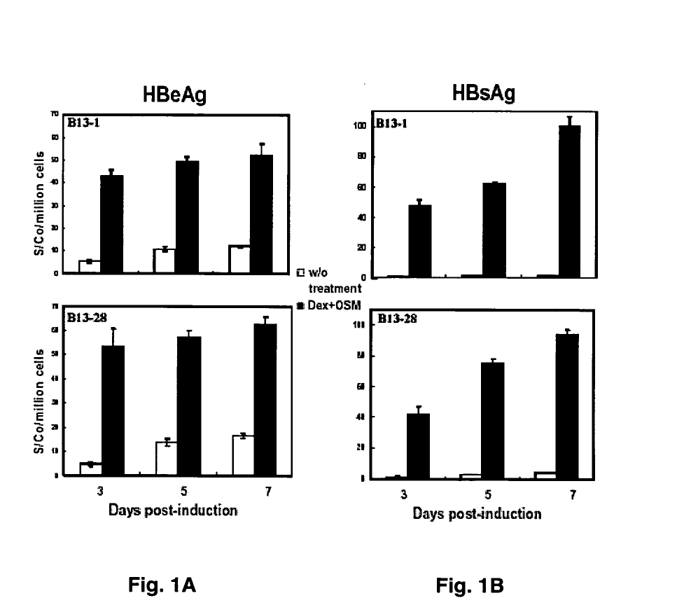

Media from B13-1 and B13-28 cells were collected on days 3, 5, and 7 after induction and were subjected to ELISA assay for both HBsAg and e antigen (Tai et al., 2002) (FIG. 1). Total cell number counts from each dish were used for normalization of the ELISA readings. As shown in FIG. 1A, e antigen secretion after induction in both B13-1 and B13-28 cells were increased by approximately 8 fold on day 3 and 4-5 fold on day 7. Surprisingly, HBsAg titer was increased by 40- to 100-fold upon induction in both B13-1 and B13-28 cells (FIG. 1B). The 2-fold increase of HBsAg from day 3 to day 7 probably reflected a doubling of cells converted to hepatocyte-like cells (FIG. 1B).

### EXAMPLE 3

Confocal Immunofluorescence Staining of HBV Core Antigen (HBcAg) and a Liver Marker

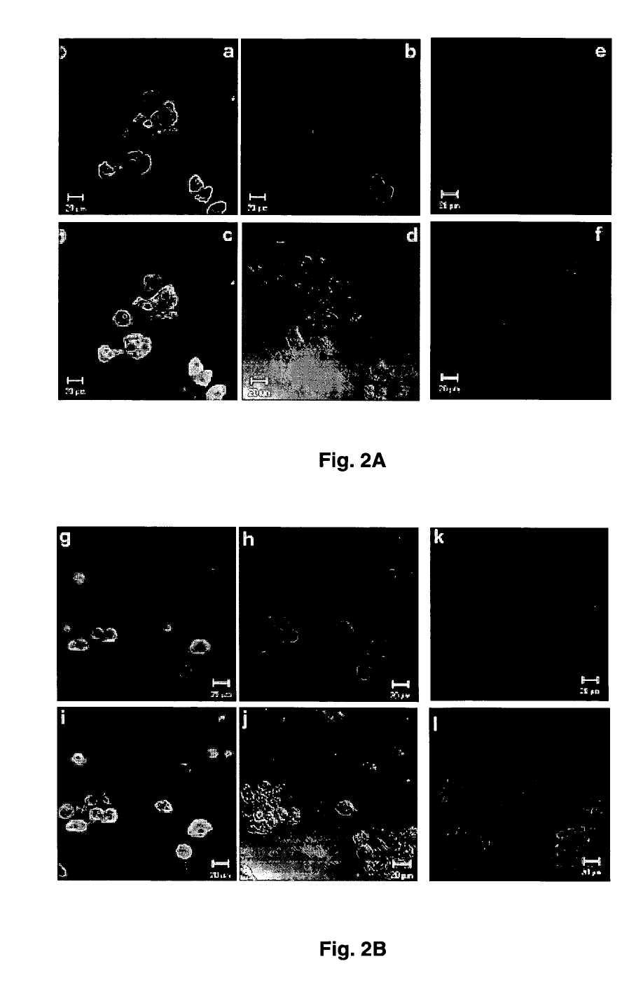

Intracellular expression of HBV core antigen (HBcAg) and liver specific glutamine synthetase were examined in B13-1 (FIG. 2A) and B13-28 cells (FIG. 2B).

For immunofluorescent staining, cells were cultured on noncoated glass coverslips, rinsed with PBS twice, fixed with 4% paraformaldehyde in PBS for 30 min at room temperature, then permeabilized with 0.1% (v/v) Triton X-100 in PBS for 30 min and incubated in 2% blocking buffer (Roche) for 1 hr. The cells were then incubated sequentially with primary and secondary antibodies (Table 1). Coverslips were incubated with DAPI (500 ng/ml in PBS) for 5 min at room temperature. After immunostaining, the coverslips were mounted on slides in gelvatol medium (20% polyvinyl alcohol in 10 mM Tris-HCl, pH 8.6). Images were collected using a Zeiss confocal microscope (LSM 510) and processed with PHOTOSHOP.

As expected, un-induced cells did not express HBcAg or glutamine synthetase (right panels of FIGS. 2A and 2B). In contrast, induced cells appeared to be a heterogeneous population and expressed HBcAg (green) and glutamine synthetase (red) to various degrees. Some cells co-expressed both HBcAg and glutamine synthetase, as shown by the yellow color in overlaid images (FIGS. 2c and 2i). Yet, there were cells expressing only glutamine synthetase but no HBcAg (remained red in overlaid images). Cells expressing only HBcAg and no glutamine synthetase remained green in overlaids.

Because cytoplasmic HBcAg can be accumulated in the nucleus, green nuclei were often observed in FIGS. 2c and 2i. For reasons unclear, red nuclei were observed in FIG. 2c, indicating nuclear presence of glutamine synthetase on rare occasion. The lack of consistent co-expression of HBcAg and glutamine synthetase suggests that while the single-cell cloned populations of B13-1 and B13-28 cells were treated with dexamethasone and oncostatin M at the same time, their responses to induction were heterogeneous, at least in the expanded cell sizes (see below) and in their respective contents of liver transcription factors.

TABLE 1
Primary And Secondary Antibodies Used For Western Blot And
Immunofluorescence Analyses

| | | | Second- | | |
|---|---|---|---|---|---|
| | Dilution | Primary | ary | Western | Source |
| Antibodies | rate | Ab | Ab | IFA | (Vendor) |
| Rabbit polyclonal | 1/500 | — | | W, IFA | Dako |
| anti-HBcAg | | | | | |
| Mouse monoclonal | 1/200 | — | | IFA | Chemicon |
| anti-HBcAg | | | | | |
| Goat polyclonal | 1/300 | — | | IFA | Dako |
| anti-HBsAg | | | | | |
| Mouse Monoclonal | 1/200 | — | | W | Institute of |
| anti-HBsAg | | | | | Immunology |
| Rabbit polyclonal | 1/500 | — | | W, IFA | Sigma |
| anti-α-amylase | | | | | |
| Rabbit polyclonal | 1/500 | — | | W | Sigma |
| anti-α1-antitrypsin | | | | | |
| Rabbit polyclonal | 1/200 | — | | IFA | BD |
| anti-glutamin | | | | | Biosciences |
| synthetase | | | | | |
| Rabbit polyclonal | 1/200 | — | | IFA | Sigma |
| anti-transferrrin | | | | | |
| Rabbit polyclonal | 1/50 | — | | IFA | Pei and Shih |
| anti-C/EBP α | | | | | (1991) |
| Rabbit polyclonal | 1/200 | — | | IFA | Santa Cruz |
| anti-C/EBP β | | | | | |
| Rabbit polyclonal | 1/200 | — | | IFA | Santa Cruz |
| anti-HNF 4α | | | | | |
| Goat polyclonal | 1/200 | | — | IFA | Sigma |
| anti-mouse IgG | | | | | |
| FITC/TRITC | | | | | |
| Goat polyclonal | 1/200 | | — | IFA | Sigma |
| anti-rabbit IgG | | | | | |
| FITC/TRITC | | | | | |
| Goat polyclonal | 1/200 | | — | IFA | Sigma |
| anti-mouse IgG | | | | | |
| FITC/TRITC | | | | | |
| Donkey polyclonal | 1/200 | | — | IFA | Santa Cruz |
| anti-goat IgG | | | | | |
| FITC/TRITC | | | | | |
| Donkey polyclonal | 1/200 | | — | IFA | Santa Cruz |
| anti-rabbit IgG | | | | | |
| FITC/TRITC | | | | | |

### EXAMPLE 4

Confocal Immunofluorescence Staining of HBV Surface Antigen (HBsAG) and a Liver Marker

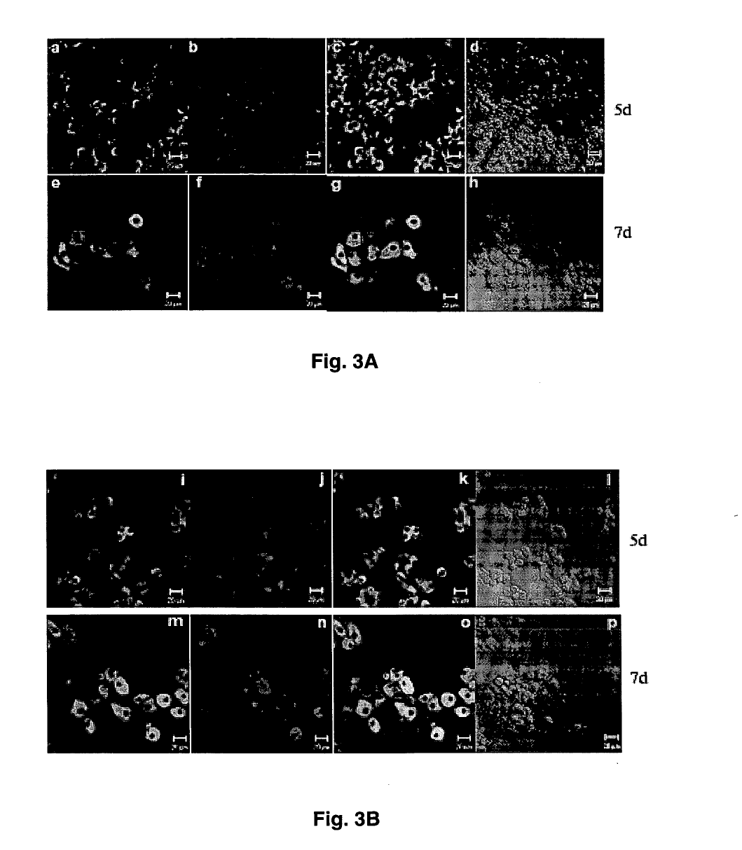

Similarly, both HBsAg (green) and transferrin (red) can be detected in B13-1 (FIG. 3A) and B13-28 cells (FIG. 3B) on day 5 or day 7 post-induction. As was the case in FIG. 2, significant degree of heterogeneity was observed in the expression of HBsAg and transferrin. There were always a substantial fraction of cells that would express only the viral marker, or only the liver cell marker, but not both. During the period between day 5 and 7, cell size expansion occurred as evident by comparing the same scale bar (20 μm) used in all pictures (FIG. 3). Consistent with in vivo situation, parental pancreatic cells tended to be smaller in size while the trans-differentiated hepatocytes tended to be enlarged in size and more flattened (FIG. 3).

### EXAMPLE 5

Co-Expression of Liver Transcription Factors and HBsAg

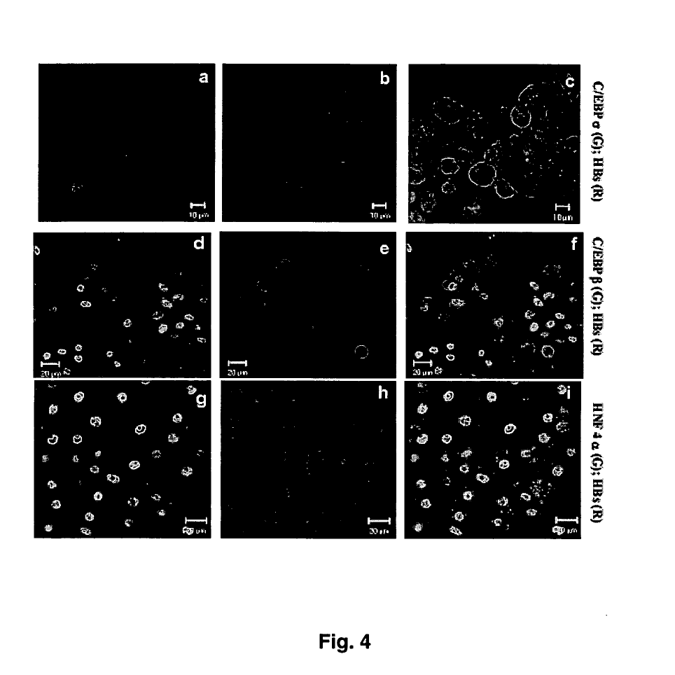

Liver gene expression is regulated by a number of transcription factors. Expression of HBsAg in the cytoplasm (red) and three liver transcription factors (C/EBP-α, C/EBP-β, and HNF4-α) in the nucleus (green) were examined in FIG. 4. The nuclei were stained blue with DAPI. Results in FIGS. 4c, 4f, and 4i indicated that HNF4-α has the best correlation with the expression of HBsAg. These transcription factors are most likely to play a direct or indirect role in the dramatic increase of HBsAg in response to dexamethasone and oncostatin M in B13-1 and B13-28 cells.

### EXAMPLE 6

Western Blot Analysis of HBcAg, Liver, and Pancreatic Markers

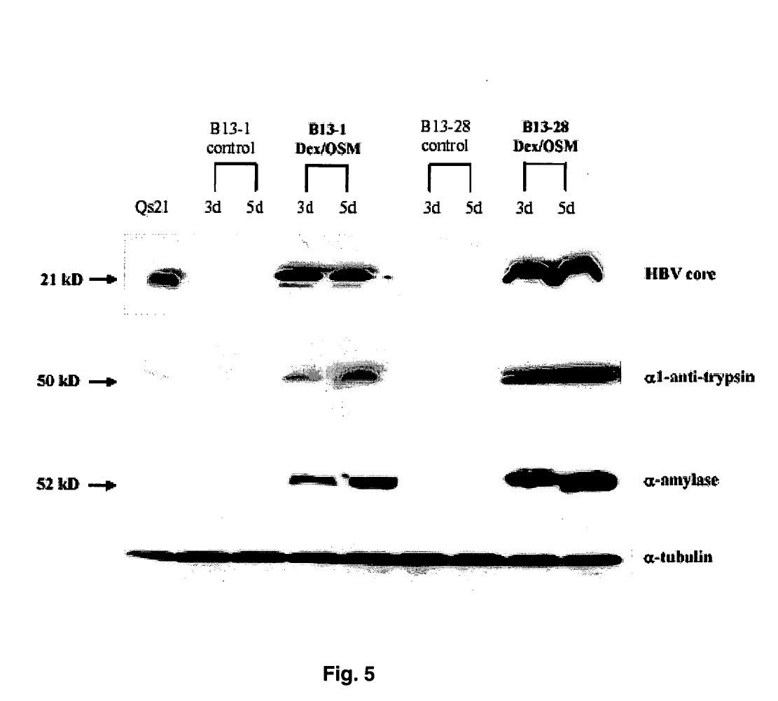

To confirm the immunofluorescence results in FIG. 2-4, Western blot analysis of B13-1 and B13-28 cells were performed before and after induction using antibodies specific for HBcAg, α1-anti-trypsin, α-amylase, and α-tubulin (FIG. 5).

As predicted, HBcAg and al-anti-trypsin were induced upon dexamethasone and oncostatin M treatment and α-tubulin was unaffected by the treatment. Intriguingly, α-amylase was undetectable in B13-1 and B13-28 cells without induction. However, α-amylase was detected in parental AR42JB13 cells even before treatment (data not shown). Therefore, B13-1 and B13-28 cells appeared to be different from their parental AR42JB13 cells in their basal level of α-amylase before induction.

### EXAMPLE 7

Analysis of HBV Specific RNAs Before and After Induction

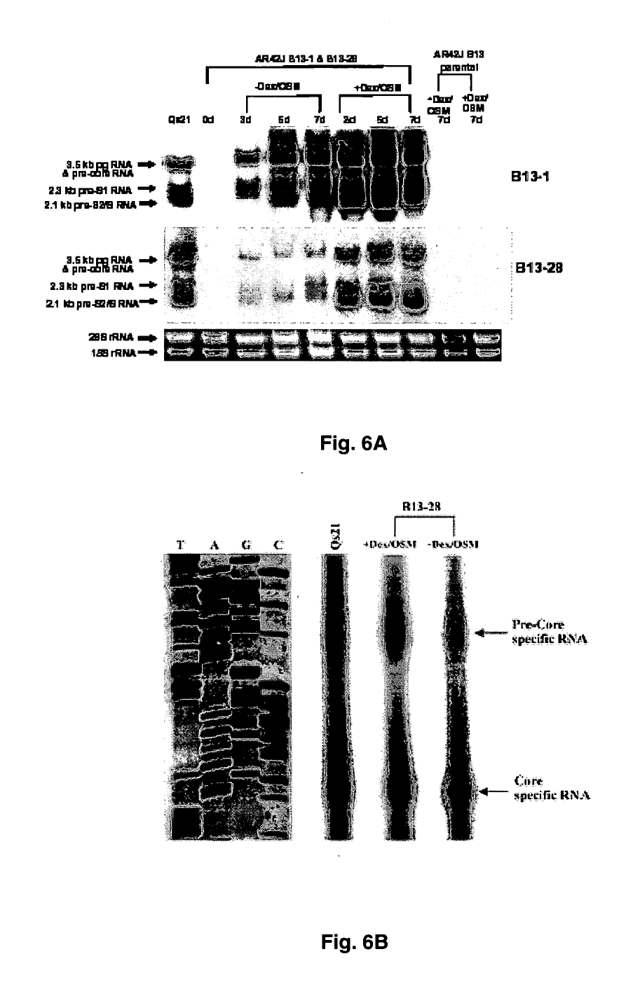

The increased protein production of HBsAg (FIGS. 1 and 3) and HBcAg (FIGS. 2 and 5) could be resulted from transcriptional stimulation by dexamethasone and oncostatin M (Dex+OSM). To examine this possibility, Northern blot analysis of total intracellular RNA of B13-1 and B13-28 cells was performed before and after induction. As shown in FIG. 6A, the most dramatic effect by dexamethasone and oncostatin M was observed in the 2.1 kb preS2/S RNA species. In contrast, no significant effect on the 2.3 kb preS1 RNA was observed.

Moderate degree of dexamethasone and oncostatin M effect was observed in the 3.5 kb RNA species, which consists of pgRNA (core-specific RNA) and pre-core RNA. These two RNA species are structurally related and can be distinguished from each other at their 5′ ends. To resolve these two closely related RNA species, primer extension analysis was performed using total intracellular RNA prepared from B13-28 cells with or without Dex+OSM. Primer extension protocol was adapted from Roychoudhury et al. (1991). Briefly, a 5′ end-labeled oligonucleotide (1930AS, 5′-GAGAGTAACTCCACA GTAGCTCC-3′, SEQ ID NO:1) was annealed at 65° C. for 10 min with core-associated RNA (from one transfected 10-cm dish) in a buffer containing 45% formamide, 1 mM EDTA, pH 7.8, 40 mM PIPES, pH 6.25, 400 mM NaCl. This mixture was then cooled down to room temperature and 20 μl of 3M NaOAc, 150 μl DEPC-treated water, and 400 μl of ethanol were added for precipitation. After centrifugation, the pellet was dissolved in 12 μl DEPC-treated water and reverse transcription was performed at 42° C. for 1.5 h with 20U of M-MuLV RT (New England Biolabs, Beverly, Mass.), 2 μl of RT buffer, 1 μl of 10 mM dNTP mixture, 2 μl of 0.1M DTT, 10U of RNasin and 1 μg of actinomycin D. The reaction was terminated by adding 1 μg of 0.5M EDTA, pH 7.8 and 1 μl of RNase A for 30 min at 37° C. A 200 μl volume of TE/0.1M NaCl was added to the reaction, followed by phenol/chloroform extraction and ethanol precipitation. After centrifugation, the pellet was dissolved in 3 μl TE, 3 μl of sequencing loading buffer was added, and run on a 6% polyacrylamide sequencing gel.

As shown in FIG. 6B, core specific RNA was not stimulated by induction while the pre-core RNA displayed an approximately 3.5-fold induction. Qs21 is a known HBV-producing cell line and its RNA was included as a positive control. Because pre-core protein is the precursor to e antigen, the results in FIG. 6B is consistent with the ELISA data in FIG. 1.

### EXAMPLE 8

Southern Blot Analysis of HBV DNA Replication Before and After Induction

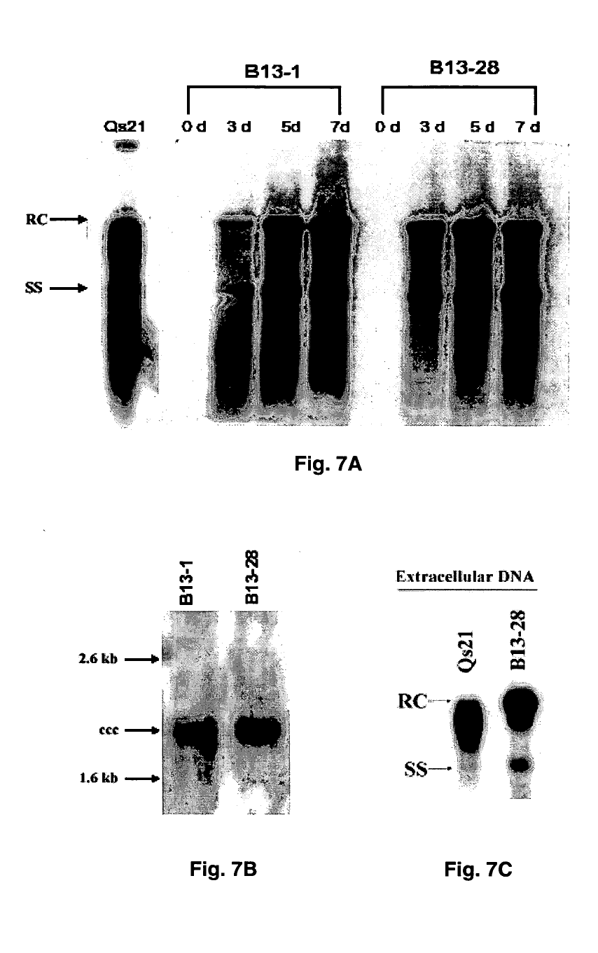

Although B13-1 and B13-28 cells are capable of expressing hepatitis B virus RNA and protein upon induction (FIG. 1-6), it remains unclear if hepatitis B virus can indeed perpetuate itself in this system. To address this issue, Southern blot analysis of viral DNAs prepared from B13-1 and B13-28 cells before and after induction was conducted. HBV DNA isolated from Qs21 was included again as a positive control. As shown in FIG. 7A, characteristic replication patterns of hepatitis B virus DNA was observed, including full-length single strand (SS) and relaxed circular (RC) replicative intermediates on day 3, 5, and 7 post-induction. It was noted that the full-length RC form in this system tends to be more abundant than other HBV-producing systems, such as Qs21.

HBV replicates via an RNA intermediate. The pre-genomic RNA is transcribed from the covalently closed circular (ccc) DNA template in the nucleus. However, in B13-1 and B13-28 cells, the HBV tandem dimer plasmid is most likely integrated into the host chromosomes during transfection. Such integrated copies could in theory serve as a substitute for ccc DNA and engage in transcription of pgRNA.

To see if ccc DNA can be found in B13-1 and B13-28 cells after induction, ccc DNA was analyzed by Southern blot analysis. For the isolation of covalently closed circular (ccc) DNA, cells were lysed with 0.5% Nonidet P-40 and the nuclei were collected by low-speed centrifugation (5,000 rpm for 5 min). Covalently closed circular DNA-containing samples were diluted in an equal volume of 0.1 N NaOH and incubated at 4° C. for 10 min. to irreversibly denature non-covalently closed, double-stranded DNA species. The DNA was neutralized by adding 3 M potassium acetate (pH 5.2) to a final concentration of 0.6 M. Single-strand DNA was efficiently removed at this pH by phenol extraction. Double-strand ccc DNA remaining in the aqueous phase was recovered by ethanol precipitation.

As shown in FIG. 7B, HBV specific signal can be detected at a position expected for ccc DNA. Restriction enzyme EcoRI is known to cut once in the hepatitis B virus (ayw subtype) genome. When the extracted ccc DNA preparation was pre-digested with EcoRI before loading, this putative ccc DNA band upshifted to a 3.2 kb position on the 1.2% agarose gel, which is where a linearized full-length hepatitis B virus genome would band (data not shown).

To see if intracellular hepatitis B virus capsids can be enveloped and secreted into the medium of B13-1 and B13-28 systems, virions were analyzed by density gradient centrifugation (data not shown). Fractions corresponding to the expected density of hepatitis B virus virions (around 1.24 g/cm³) were collected and dialyzed to remove CsCl. Virion-associated DNA was extracted and analyzed by Southern blot. As expected from wild type hepatitis B virus, mature genome was preferentially exported (FIG. 7C).

### EXAMPLE 9

Electron Microscopic Examination of Secreted HBV Viral and Subviral Particles in the Medium of Trans-Differentiated B13-28 Cells

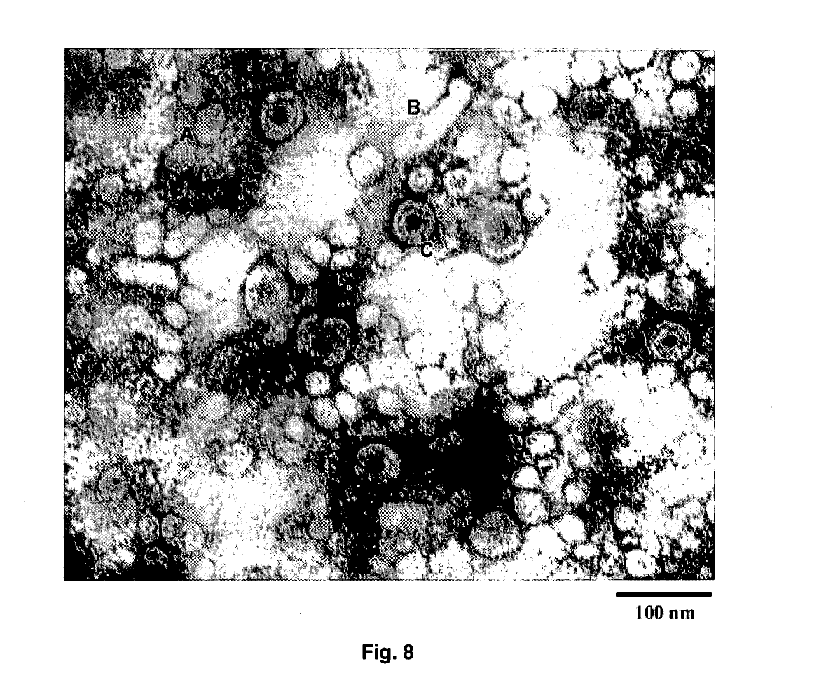

Results shown in FIG. 7C was confirmed by using a standard procedure for transmission electron microscopy. As shown in FIG. 8, in addition to the tubular and spherical subviral particles, Dane-like particles with an electron dense core, concentric shape, and approximately 42 nm in diameter were detected. This result suggests that the hepatocyte-like cells of trans-differentiated B13-1 and B13-28 can indeed support virion assembly and secretion of mature genome similar to an authentic liver cell.

### EXAMPLE 10

Continuous Presence of Dexamethasone and Oncostatin M is Required For HBV Replication and Gene Expression In B13-1 and B13-28 Cells

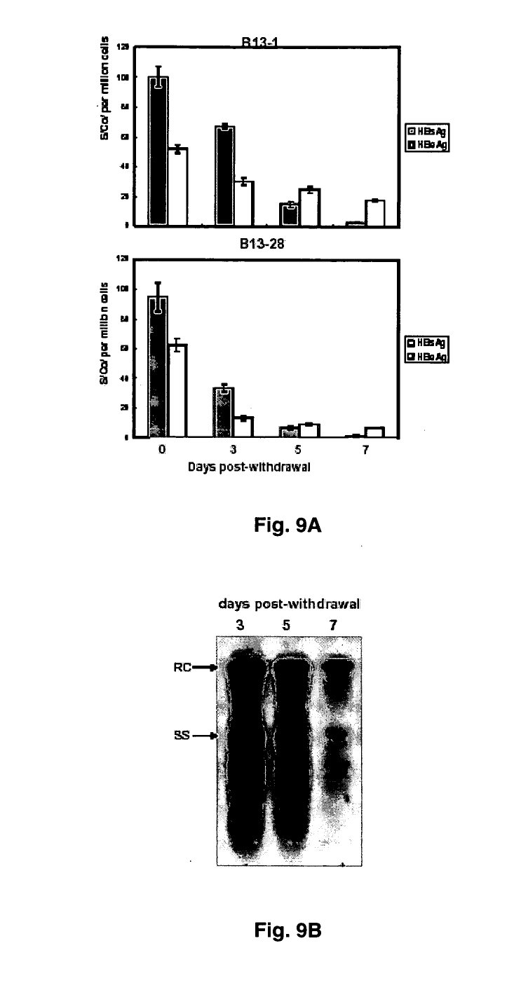

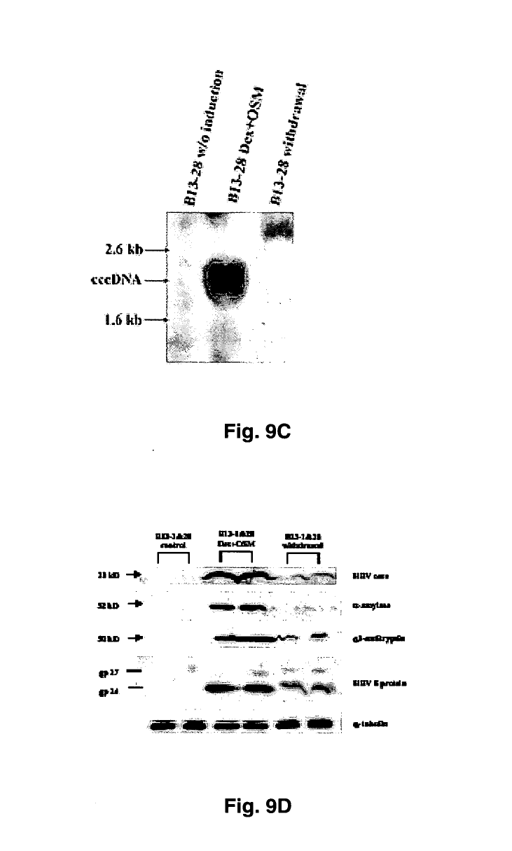

When dexamethasone and oncostatin M were removed from the medium, the levels of secreted HBsAg and e antigen (FIG. 9A), and RC and SS DNA decreased rapidly (FIG. 9B). Similarly, covalently closed circular DNA dropped to almost undetectable level by day 7 (FIG. 9C). Consistent with the ELISA results in FIG. 9A, intracellular HBsAg (p24 and gp27) and HBcAg decreased significantly (FIG. 9D). Finally, the decrease of viral replication and gene expression were also paralleled by the decrease of α1-anti-trypsin and α-amylase measured by Western blot analysis (FIG. 9D). Again, α-tubulin remained unaffected by dexamethasone withdrawal.

The following references were cited herein:
Mashima et al., Formation of insulin-producing cells from pancreatic acinar AR42J cells by hepatocyte growth factor. Endocrinology 137: 3969-3976 (1996).
Shen et al., Molecular basis of trans-differentiation of pancreas to liver. Nat. Cell Biol. 2: 879-887 (2000).
Shih et al., In vitro propagation of human hepatitis B virus in a rat hepatoma cell line. Proc. Natl. Acad. Sci. USA 86: 6323-6327 (1989).
Tai et al., Virology 292: 44-58 (2002).

1. A method of screening for a drug which affects the propagation and replication of hepatitis virus, comprising the steps of:
trans-differentiating a cell by a glucocorticoid steroid or a glucocorticoid steroid plus an IL-6 homologue, thereby generating a trans-differentiated cell capable of propagating and replicating hepatitis virus DNA;
contacting the trans-differentiated cell with a candidate compound, wherein inhibition of DNA replication or RNA transcription of hepatitis virus in the presence of the compound indicates that the compound has anti-hepatitis virus activity.

2. The method of claim 1, wherein the glucocorticoid steroid is dexamethasone.

3. The method of claim 1, wherein the IL-6 homologue is oncostatin M.

4. The method of claim 1, wherein the trans-differentiated cell is capable of secreting hepatitis B surface antigens and hepatitis B e/core antigens.

5. The method of claim 1, wherein the hepatitis virus is hepatitis B virus or hepatitis C virus.

6. The method of claim 1, wherein the trans-differentiated cell is selected from the group consisting of rat AR42J-B13-1 cells, AR42J-B13-10 cells, AR42J-B13-18 cells, and AR42J-B13-28 cells.

7. A transdifferentiated cell capable of propagating and replicating hepatitis B or hepatitis C virus DNA.

8. The transdifferentiated cell of claim 7, wherein said cell is induced to differentiate by a glucocorticoid steroid or a glucocorticoid steroid plus an IL-6 homologue.

9. The transdifferentiated cell of claim 8, wherein said glucocorticoid steroid is dexamethasone and said IL-6 homologue is oncostatin M.

10. The transdifferentiated cell of claim 7, wherein said cell is capable of secreting hepatitis B surface antigens and hepatitis B e/core antigens.

11. The transdifferentiated cell of claim 7, wherein said cell is selected from the group comprising rat AR42J-B13-1 cells, AR42J-B13-10 cells, AR42J-B13-18 cells, and AR42J-B13-28 cells.

12. A method of transdifferentiating a first cell into a second cell capable of propagating a virus, comprising the step of contacting said cell with a glucocorticoid steroid with or without an IL-6 homologue.

13. The method of claim 12, wherein said first cell is a pancreatic cell.

14. The method of claim 12, wherein said second cell is a hepatic cell.

15. The method of claim 12, wherein said virus is hepatitis B virus.

16. The method of claim 12, wherein said glucocorticoid steroid is dexamethasone.

17. The method of claim 12, wherein said IL-6 homologue is oncostatin M.
